(19) United States
(12) Patent Application Publication
(10) Pub. No.: US 2025/0029695 A1
(43) Pub. Date: Jan. 23, 2025

(54) Personal Health Care System with Improved Recordkeeping between Health Care Organizations

(71) Applicant: Shanthakumari Raju, CAVE CREEK, AZ (US)

(72) Inventor: Shanthakumari Raju, CAVE CREEK, AZ (US)

(21) Appl. No.: 18/909,516

(22) Filed: Oct. 8, 2024

Related U.S. Application Data
(63) Continuation-in-part of application No. 17/086,429, filed on Nov. 1, 2020, now abandoned.
Application No. 17/086,429 is a continuation-in-part of application No. 15/344,401, filed on Nov. 4, 2016, now abandoned.
Application No. 15/344,401 is a continuation-in-part of application No. 14/481,301, filed on Sep. 9, 2014, now abandoned.

(51) Int. Cl. G16H 10/65; G06F 21/62

(52) U.S. Cl. CPC G16H 10/65; G06F 21/6245

(57) ABSTRACT

The embodied invention is a personal health card with a magnetic strip or embedded chip that is carried by a person, so that in case of health emergencies or when visiting a doctor's office, the card is used to retrieve health information from a medical record website. The health information aids in obtaining proper medical treatment under a variety of conditions. The personal health card works with an interactive software program where a registered user updates their online personal health records, and then retrieves it via the personal health card when interacting with a medical practitioner. Computer program algorithms provide improved recordkeeping between health care organizations

### CROSS REFERENCE TO RELATED APPLICATIONS

This invention is a continuation in part of Ser. No. 17/086,429 filed on Nov. 1, 2020 which is a continuation in part of U.S. patent application Ser. No. 15/344,401 filed on Nov. 4, 2016, which is a continuation-in-part of U.S. patent application Ser. No. 14/481,301 filed on Sep. 9, 2014. The prior applications are all incorporated by reference herein.

### STATEMENT OF FEDERALLY SPONSORED RESEARCH OR DEVELOPMENT

Not applicable.

### BACKGROUND OF THE INVENTION

### (1) Field of the Invention

This invention is directed to the secure storage and communication of information related to an individual's medical history, current medical conditions, current medications, and symptoms.

### (2) Description of Related Art

Most individuals want personal health conditions and related information kept private. frequently, individuals do not want to discuss medical conditions with family members or friends, preferring to discuss them only with a medical person. physicians in particular, as well as fire fighters and other emergency responders, provide medical treatment and have a need to understand an individual's medical picture in order to provide proper treatment.

The reasons that some individuals are reluctant to share their medical conditions are many. Some may be shy and sensitive, and others may not want to bother family members or friends with bad news. Some conditions may be serious, and that person may want to avoid discussing their situation to prevent others from treating them differently. Some individuals want to avoid sympathy and be treated normally. Some may want their careers to proceed normally, especially if the treatment should not affect their work.

Consequently, when a person is unconscious or unable to respond when that information is needed in a medical setting, there is a clear need for a mechanism for that information to be readily available. Additionally, it is important that the information is accurate and kept up to date. The dependence on the memory of family members may cause problems for medical personnel.

Hospitals and other medical providers routinely do not have current health information on individuals, and it is a common practice for a medical provider to have a patient fill out an information form when an individual visits their office for treatment. The individual may not remember their medical history very well, and important dates and the use of proper medical terminology may be poorly conveyed.

It is unfortunate that there is no common database on an individual's health history and current health conditions. For example, for a receiving doctor to obtain needed information from a sending doctor, the individual must approve the transmission, and the sending doctor must send the transmission. Usually, a fax transmission is used. Such requests can pile up, especially if the medical matter is not urgent, and delays are inevitable as it often requires the doctor or office manager to personally approve sending it. It is well known that doctors are very busy.

There is a health practice that important information is affixed to the front door of a house or bedroom, such as a do not resuscitate form. This practice, especially for senior citizens with medical directives, is subject to a variety of issues. It tends to be updated infrequently, if at all, and it is not designed to list current medical conditions that will aid an emergency responder in providing suitable treatment. The posted information may be lost in an emergency, or simply forgotten in the rush to transport an individual to a hospital. In a non-emergency situation, the individual who posts the information may be unable (or incapable) of conveying the information to a medical care facility or a medical practitioner.

It is important that the information is not lost or destroyed when an emergency situation occurs. Fires, tornadoes, vehicle accidents, etc. all cause the need for the information to rise, and at the same time, increase the likelihood that the information will be lost. It is critical that the information is kept securely in a manner that reasonably will not be lost in catastrophe situation.

### BRIEF STATEMENT Of THE INVENTION

The embodied invention is a personal health card with a magnetic strip or embedded chip that is carried by a person, so that in case of health emergencies or when visiting a doctor's office, the card is used to retrieve health information from a medical record website. The health information aids in obtaining proper medical treatment under a variety of conditions. The personal health card works with an interactive software program where a registered user updates their online personal health records, and then retrieves it via the personal health card when interacting with a medical practitioner. AI algorithms provide improved recordkeeping between health care organizations

### BRIEF DESCRIPTION OF THE FIGURES

FIG. 1 is an example personal health card.

FIG. 2 is an example welcome screen.

FIG. 3 is an example new patient registration flow diagram.

FIG. 4 is an example of how to manage patient information.

FIG. 5 is an example patient registration data entry page.

FIG. 6 is an example patient registration thank you page.

FIG. 7 is an example patient profile (first time) page.

FIG. 8 is an example registered user sign in page.

FIGS. 9-10 is an example new authorized user registration flow diagram.

FIG. 11 is an example sign in page for an authorized user (on behalf of patient).

FIG. 12 is an example patient login page.

FIG. 13 is an example patient information management page, used by a patient or

an authorized user.

FIG. 14 is an example authorized user/patient one-time password page.

FIG. 15 is an example new health care provider registration flow diagram.

FIG. 16 is an example new health care provider registration page.

FIG. 17 is an example new health care provider thank you page.

FIG. 18 is an alternate example health care provider registration page without a one-time password.

FIG. 19 is an example unregistered health care provider access flow diagram.

FIG. 20 is an example unregistered health care provider registration page.

FIG. 21 is an example is an unregistered health care provider one-time password request.

FIG. 22 is an example unregistered health care provider patient access page.

FIG. 23 is an example patient health symptoms update.

FIG. 24 is an example patient health history page, utilizing an improved patient interface.

FIG. 25 is an abbreviated view of connections between the user, health care practitioner and the medical record database.

FIGS. 26A-26B show a phone lock screen with an ‘in case of emergency’ contact name and a phone number.

FIG. 27 is a simplified block diagram of the patent intake process.

FIG. 28 is a simplified block diagram of the emergency medical process.

FIG. 29 is a simplified block diagram of the Patient GPS Monitoring.

FIG. 30 is a block diagram of important of AI Enhanced Doctor and Hospital Recommendations.

### DETAILED DESCRIPTION OF THE INVENTION

The disclosed invention is a personal health card that is connected to an online record of their medical history as well as current health symptoms. Both users and health care providers are asked to register with a personal health website and keep the information current and accurate. In particular, the online record includes any health concern that is worrisome to a user and important to communicate to medical providers during any emergency.

After successfully registering on a personal health website, a registered user receives a plastic card in the mail with an embedded magnetic strip or readable microchip. It is preferable to utilize a microchip or a magnetic strip rather than print a registration number or identification label on the card. This provides a basic security barrier to prevent unwelcome individuals from accessing the registered user's personal information.

Once the card is received by a registered user, it is activated by using a phone activation procedure, a reply in the mail, or by logging onto the personal health website.

At that time, the registered user is offered the opportunity to update their medical information in the appropriate web pages and data fields. Optionally, the user verifies that the correct personal health card has been sent by typing in their phone number to an online web registration page.

In use, the registered user's online information is linked to the personal health card by use of the micro-chip or magnetic strip embedded in the card. Upon successful registration, the link between the personal health card is immediately established by the registration number embedded in the card. The microchip or magnetic strip contains the registration number that can only be read by a magnetic strip or microchip reader.

After registering, the user is responsible to log into the medical record website and keep their medical information current. Additionally, health care providers are given the opportunity to update the medical information of the registered user if they, in turn, register with the medical record website.

To update health information on the medical record website, the registered user logs in to their personal health account on the medical record website. The log in requires a user ID and a password.

When a HCP scans the personal health card, and logs into the medical record database, the HCP gets important medical information about the patient. The information includes, but is not limited to, name, birth date, address, emergency contact information, allergies, recent health history, current symptoms, and current medications; and when the last update occurred.

Typical medical items a registered user will update include: medications, prior procedures, current symptoms, amount of pain and where, tests done, allergies, a current photograph, and blood type. Typical personal identifying information includes first and last name and contact information.

Preferably, the health care provider (HCP) is a registered HCP. To become registered, the HCP goes through a login process where the address of the HCP is verified by use of a mobile phone that is capable of receiving texts.

If the HCP is unregistered, the HCP scans the patient's card number and logs into the medical record website as an unregistered HCP. The HCP must also include a phone that is capable of receiving a text or email. The HCP is then given a one-time password (a randomized alphanumeric text) via text or email for use in logging into the medical record website. The HCP then uses the personal health card number and OTP to log into the medical record website. This registration system provides for tracking the health care provider.

Similarly, the unregistered HCP is given a web link to the individual's online medical file.

If a user's personal health account is accessed by someone other than the registered user, such as a HCP, the party inquiring about the health records is recorded in a log. The user has access to this information to verify authorized access.

In an embodiment of the invention, a help desk is available for a patient who will update the medical records for a patient that is unable or unwilling to do it themselves. A toll free number is provided on the personal health card to facilitate this. The help desk is available to help enter health data, provide a new personal health card, and answer questions.

For user password difficulties, the help desk is authorized to generate a onetime password and send it to the patient's mobile phone which is used to login. The user then updates their password and is given the opportunity to update their health record.

In case of emergencies when the registered user is not in a state to communicate to their physicians, fire fighters, emergency medical responders, etc., then the personal health card is all that is needed for the HCP to obtain the patient's online medical information. By using a card reading device which scans the registration number, and visually reading other information printed on the card, a HCP will be able to access the medical record website.

Additionally, a mobile phone will include an ‘in case of emergency’ (ICE) phone number add a name that displays on the phone when it is locked. This is an important addition to improved communications by providing an alternate way to find out important medical information for the initial treatment. Alternately, the emergency responder may swipe the phone screen to bring up the phone keypad for an emergency call and key press ‘423’ (keypad alpha-numeric for ICE) to scan the phone to obtain the emergency phone number and contact name. The responder can then use the phone to call the emergency contact individual. Optionally, the responder can use the locked phone to call the emergency contact individual.

It is the registered user's responsibility to carry their personal health card with them all the time.

Updating the user's medical records is by use of an interactive, user friendly, web based (html) interface.

An important result of the medical card and medical record database is improved communication between health care providers and the patient. The patient does not have to fill out a new form and remember their medical history each time they visit a HCP. And the information will be readily available for emergency responders, primary care providers, specialists, surgeons, and medical facilities.

The patient registration number on the magnetic strip or readable chip is designed to be readable by any card reading device already in the market.

A plastic card has a better chance of surviving potential catastrophic events, such as a car accident or a natural disaster, because it will normally be protected inside a purse or wallet.

A web page is a document that is suitable for the World Wide Web and web browsers. A web browser displays a web page on a monitor or mobile device. The web page is what displays, but the term also refers to a computer file, usually written in HTML or comparable markup language. The web page is also interactive and receives user input; and responds by recording entered data or taking action in some way. A website is a location connected to the Internet that maintains one or more web pages on the World Wide Web.

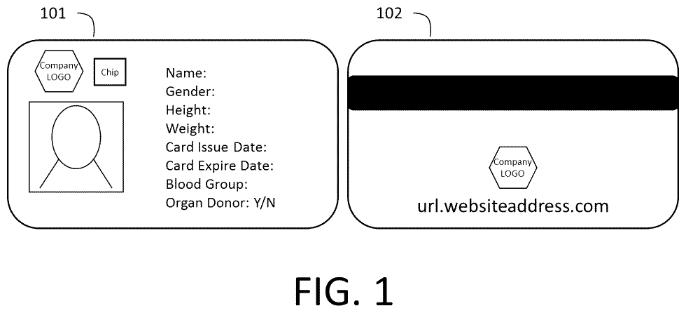

FIG. 1 is an example personal health card. The front of the personal health card 101 is shown and includes a user picture, a readable chip with the user registration number, a company logo that tells the users that it is a personal health card, and personal contact information. The blood group and organ donor are optionally shown on the front of the card. The back of the personal health card 102 shows a magnetic strip that holds the user registration number.

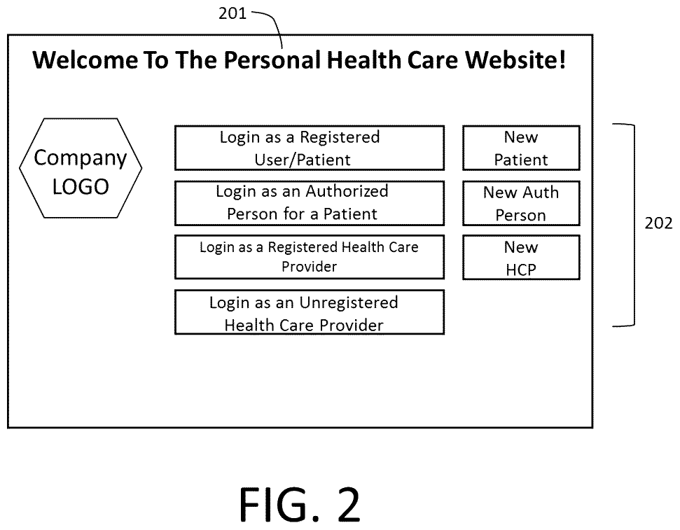

FIG. 2 is an example user welcome page. A welcome message 201 is shown at the top of the web page to confirm that the correct page has been reached. The user is given various selections 202 to click on, including: login as a registered user/patient, new patient, login as an authorized person for a patient, new authored person, login as a registered health care provider, new health care provider, and login as an unregistered health care provider.

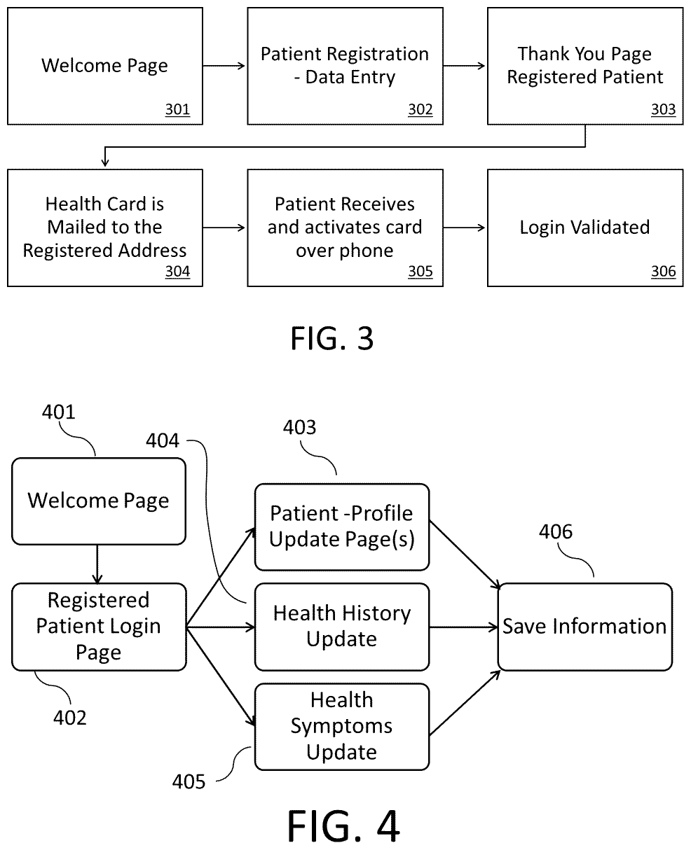

FIGS. 3-4 are an example new patient registration flow diagram.

The patient starts at a welcome screen (i.e. FIG. 2)

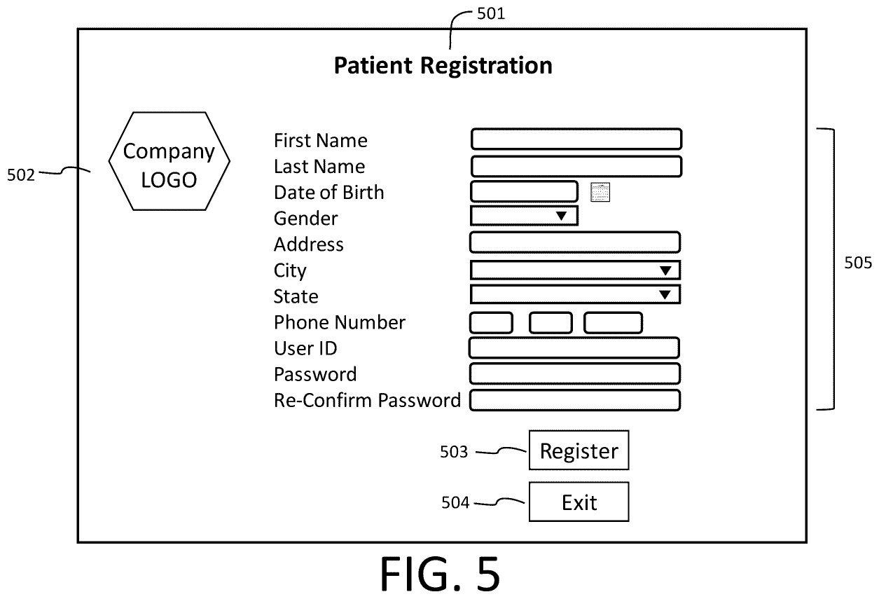

The patient then proceeds to a Patient Registration screen for entering basic contact data, such as seen in FIG. 5, to obtain a Health card.

The patient then arrives at a thank you/notification page that the patient is registered.

A health card is then mailed to the patient address entered in the contact data

The patient receives the health card and activates it over the phone

The patient registration (login access to website) is validated by a phone message, and the patient has completed the basic registration process.

FIG. 4 shows how a registered patient can update health records.

### Welcome Page

The patient arrives at a welcome registered patient login page and then can update patient information by three options:
to update the patient profile
to update health history, or
to update health symptoms

When completed, the patient is then given the opportunity to save the updated information

FIG. 5 is an example patient registration page. A patient registration message 501 is shown at the top of the web page to confirm that the correct page has been reached. A company logo 502 is included for web page branding, and to further assure the user that the process is supported by a company. A register click button 503 is included to provide for sending the data that has been inserted into the data fields 505 to the medical record database. An exit button 504 is provided to return to the home/welcome page.

If a patient wants to have a personal health card but does not know how to register on the medical record website, the patient will have to obtain his/her own assistance from family members and friends. Alternately, the patient may request help from a HCP who is willing to assist.

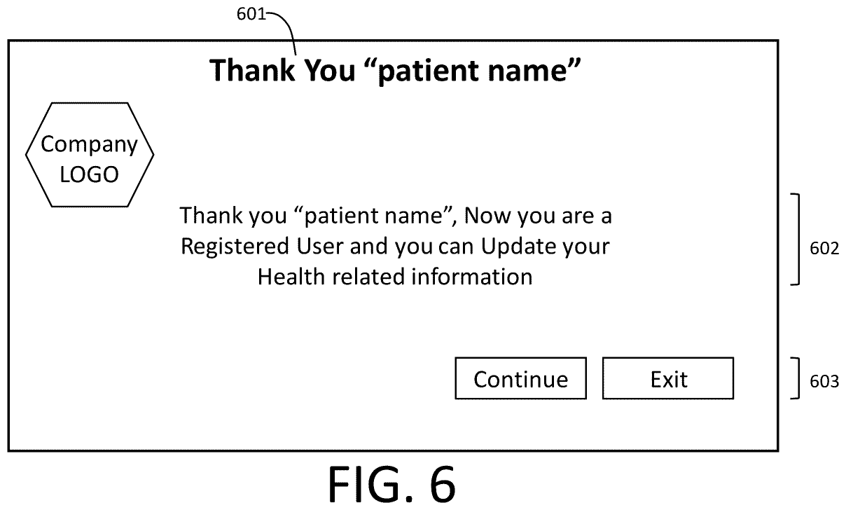

FIG. 6 is an example user thank you page. A patient thank-you message 601 is shown at the top of the web page to confirm that the correct page has been reached. Information for the next step 602 in the process is shown, and continue/exit buttons 603 all the user to ‘continue’ by entering the medical health record update page or to ‘exit’ to the home welcome screen.

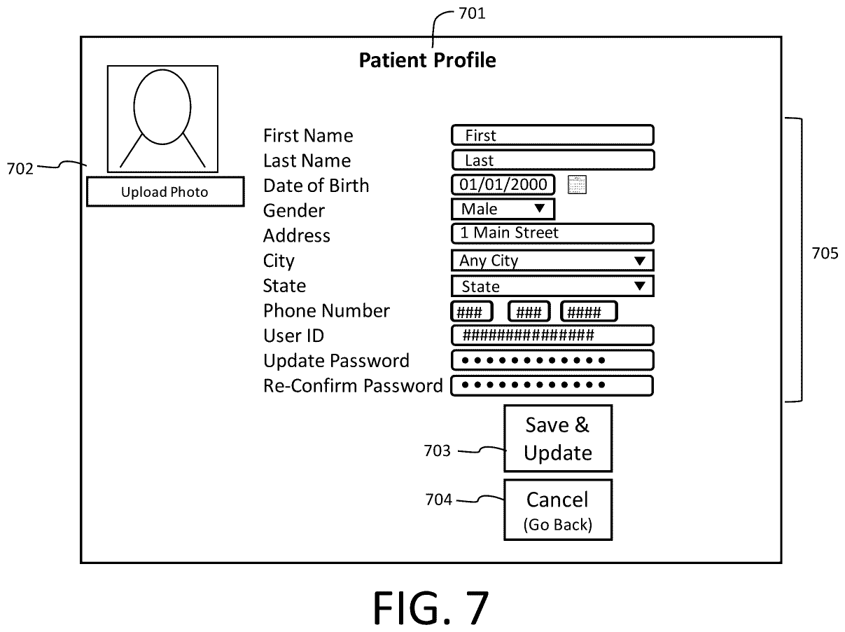

FIG. 7 is an example update and save patients profile page. A patient profile message 701 is shown at the top of the web page to confirm that the correct page has been reached. The photo on file is shown with an upload new photo button 702 to provide for updating the photo from a picture on a computer storage device or mobile device. The users current profile information is shown 705 or is blank if filling in for the first time. The user can then update the information and click on the save and update button 703. The user can click on a cancel button 704 and return to the previous page.

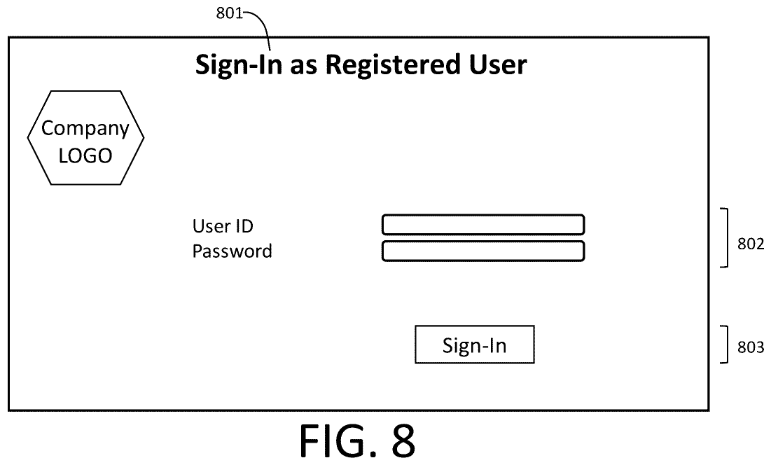

FIG. 8 is an example user sign-in page. A sign-in as a registered user message 801 is shown at the top of the web page to confirm that the correct page has been reached. Two data fields 802 are used for login, a user identification (name) and a password. The user fills in the data fields and clicks the Sign-In button 803. The user is then taken to the medical update page where they can update their profile information and health records. The health records are dynamic in the sense that they are updatable, and also, can be added to or deleted.

Both HCPs and patients (i.e. users) are encouraged to register with the medical record website, so both parties have the advantages of improved medical communication. A separate login for a HCP is utilized.

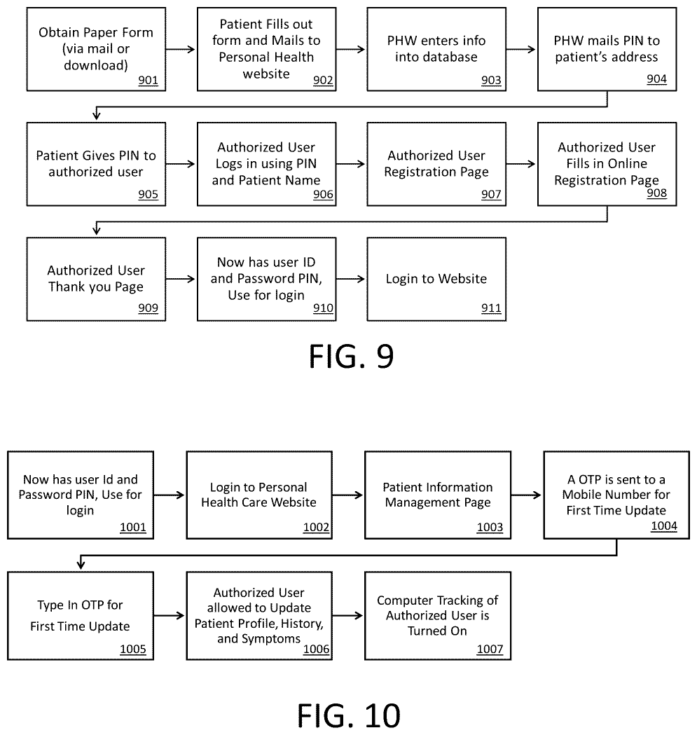

FIGS. 9 and 10 illustrates how a new authorized user (i.e. user on behalf of a patient) registers on the health care website. This is especially useful when a parent registers a child or when an adult registers their aging parent.

To become an authorized user for a patient, the authorized user and patient follow the following steps:
An authorized user obtains a paper form (via mail or download)
Patient fills out form and mail to personal health website
Personal health website enters the form information into database
Personal health website mails pin to patient's address
The patient gives the PIN to authorized user
The authorized user logs in on a login page using pin and patient name, authorized user registration page
The authorized user goes to the authorized user registration page
The authorized user fills in online registration page
The authorized user arrives at a confirming thank you page.
The authorized user now had user ID, password, and Pin to use for login
The authorized user is able to login to personal health care website

FIG. 10 shows additional steps for how an authorized user logs in to the personal care website and updates the patient information.
The authorized user now had user ID, password, and Pin to use for login
The authorized user logs into the personal health care website
The authorized user arrives at the patient management page.

When updating the patient information for the first time, a onetime password (OTP) is sent to the authorized user mobile phone number to confirm a phone number for the authorized user

For the first update, the authorized user types in the OTP.

The authorized user proceeds to update the patient health information (profile, history, and symptoms)

The personal health care website tracks the access of the authorized user and enters it to a log that is available to the patient.

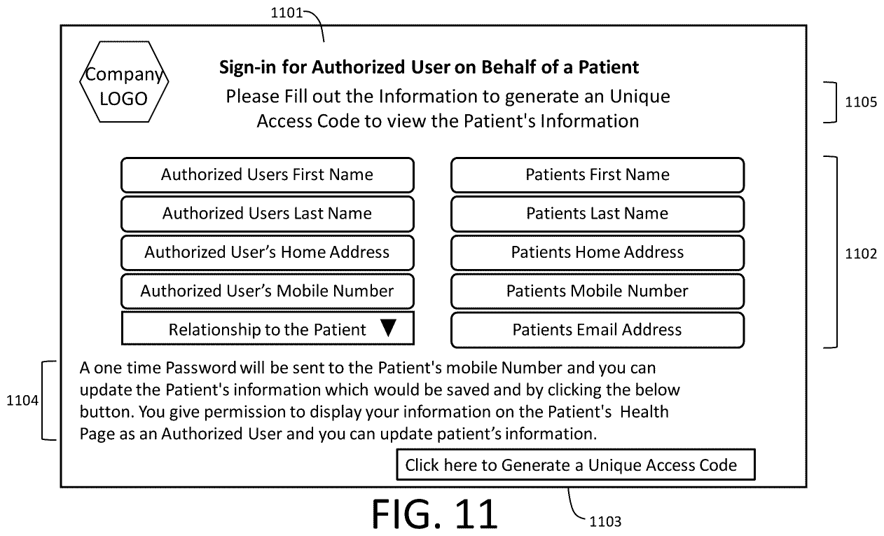

FIG. 11 is an example of where an authorized person signs in for someone else. An authorized user on behalf of a patient message 1101 is shown at the top of the web page to confirm that the correct page has been reached. An instruction message 1105 is used to inform the authorized person the goal of the page. A number of patient (i.e. user) data fields 1102 are filled in and then the authorized user clicks the access code button 1103. An instruction message 1104 tells the authorized user what to expect when access code button 1103 is clicked. The access code is sent to the authorized user's phone number. The authorized user then creates or accesses the user's personal health account.

For example, an authorized user is needed for a parent to register a child on the medical record website, or an adult who registers an aging parent.

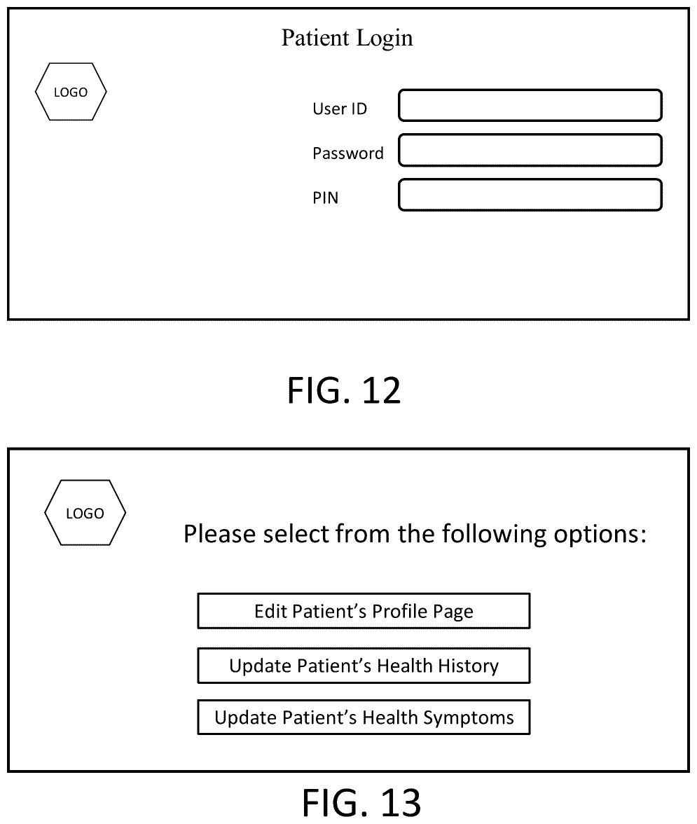

FIG. 12 is an example patient login page. The patient will need a user ID, a password, or a PIN.

FIG. 13 is an example authorized user/patient information management page. Similar to FIG. 4, how an authorized user can update a patient's health records.

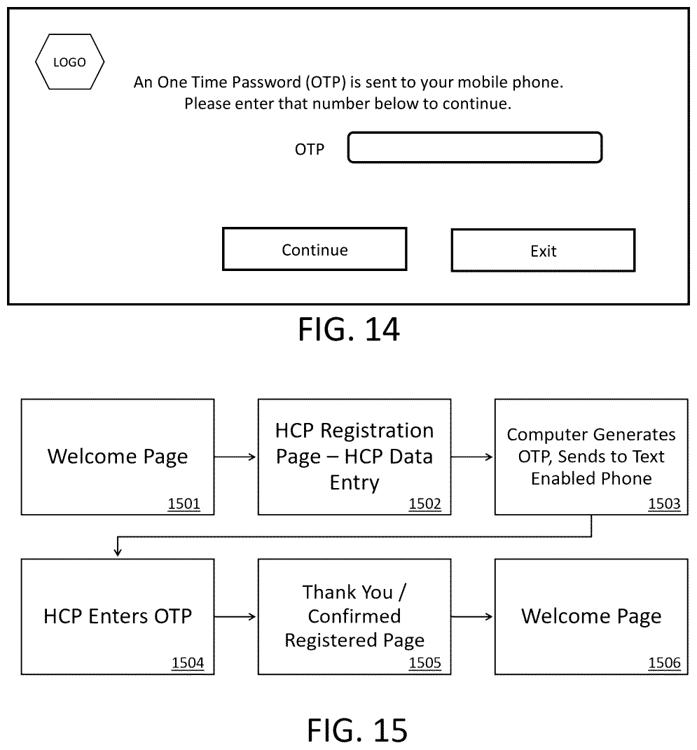

FIG. 14 is an example authorized user/patient one-time password page as would be used as explained in FIG. 10. The authorized user enters the OTP and presses the ‘Continue’ button to continue with the registration process.

FIG. 15 is an example new health care provider (HCP) registration flow diagram.

The HCP starts at a welcome page

The HCP is directed to a registration page, and inputs data about the HCP information.

The computer generates an OTP and sends it to an authorized HCP text enabled mobile phone.

The HCP receives the OTP, and enters it into an OTP web page

A thank you/confirmation page appears when the correct OTP has been entered

The HCP is directed to a welcome page

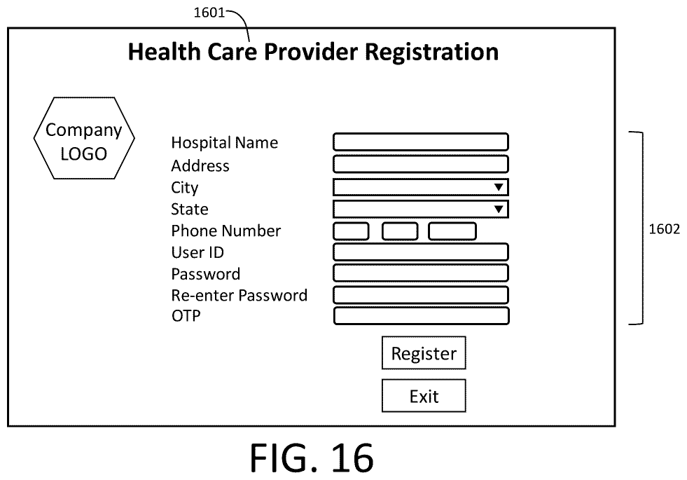

FIG. 16 is an example health care provider registration page as mentioned in FIG. 15. A health care provider registration message 1601 is shown at the top of the web page to confirm that the correct page has been reached.

The health care provider enters the data in the associated data fields 1602 and then clicks either the register or exit buttons. When registering, the health care provider receives a confirming email.

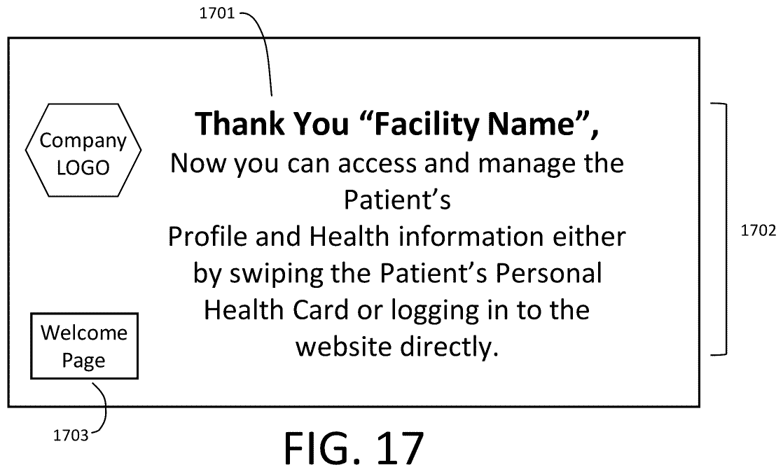

FIG. 17 is an example health care provider thank you page. A health care provider thank you message 1701 is shown at the top of the web page to confirm that the correct page has been reached. A thank you message 1702 is given to the particular hospital to confirm that the registration has been recorded. A return to the welcome page button 1703 to allow the health care provider to access additional features of the medical record website.

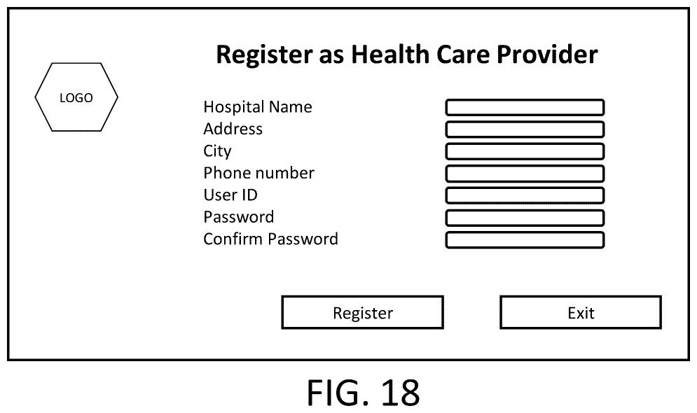

FIG. 18 is an alternate embodied registration page for a HCP without using a one-time password. It might possibly be used under certain circumstances when a separate one-time password (or other security method such as a biometric reading) is entered on a different page.

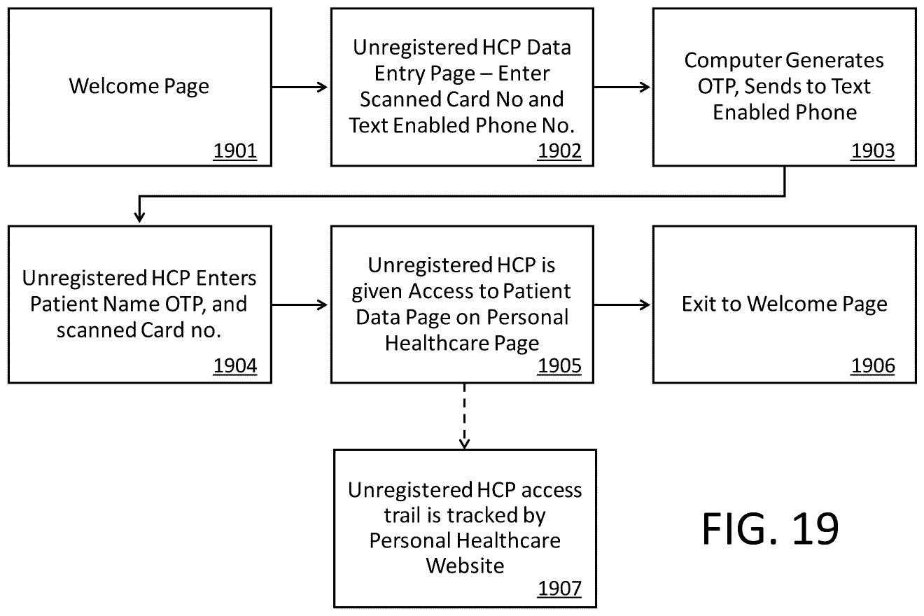

FIG. 19 is an example unregistered health care provider access flow diagram, and how the unregistered HCP obtains access to the patient information on the personal healthcare website.

The unregistered HCP starts at a welcome page.

The unregistered HCP is directed to a data entry page where the HCP enters HCP contact and identification data, scanned health card number, and the phone number of a text enabled phone

The computer generates an OTP, and sends it to the text enabled phone

The unregistered HCP enters the patient name, OTP, and the scanned health card number.

The unregistered HCP is given access to the patient data on the personal healthcare website

When done, the HCP is re-directed to the welcome page.

The HCP is then tracked by the personal health care website to record/log the access and what was viewed.

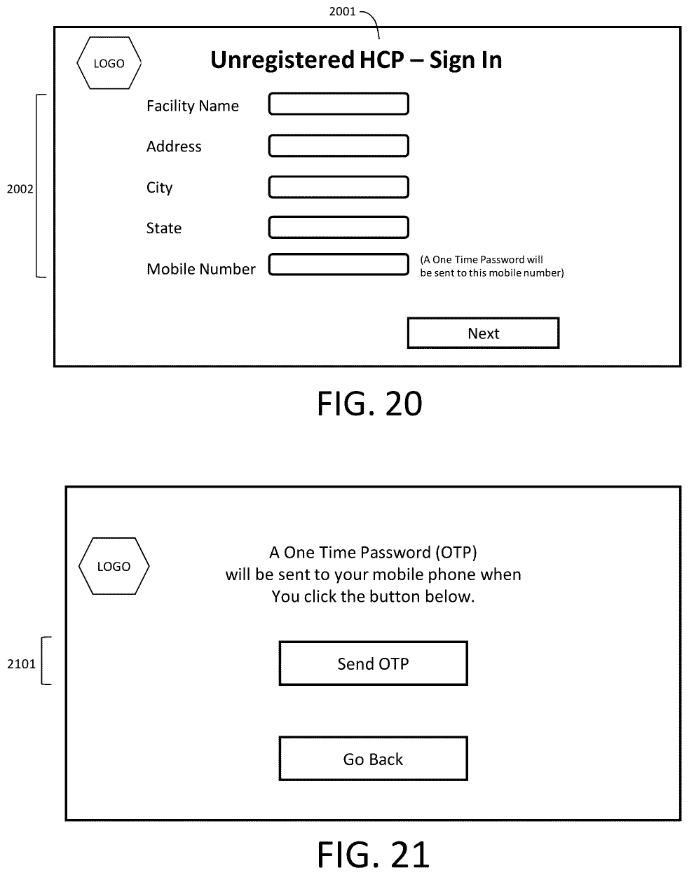

FIG. 20 is an example unregistered HCP sign in page. This page provides for a HCP to access the health records of a patient without being a registered HCP. The HCP must have access to the personal health card in order to sign in this way. The unregistered HCP—access patient information message 2001 is shown at the top of the web page to confirm that the correct page has been reached.

The unregistered HCP fills in data fields 2002 and clicks the ‘Next’ button 2003.

FIG. 21 is a follow up page to FIG. 20. The unregistered HCP clicks the ‘Send OTP’ button 2101. The unregistered user then receives a one-time password on their mobile device. Clicking on the ‘Go Back” button returns the unregistered HCP to FIG. 20.

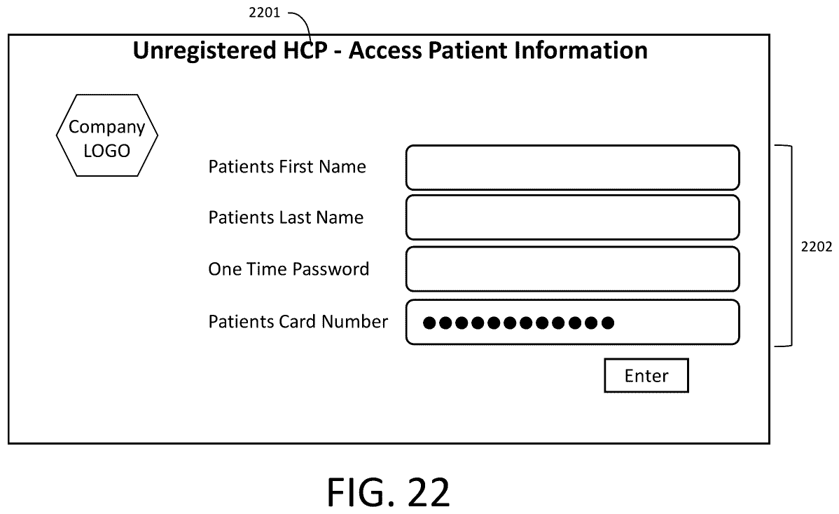

FIG. 22 shows that the HCP receives the one-time password and enters it, along with the information needed in the data fields 2202, to access to a patient's medical records. The patient's card number is automatically input to this screen via a card/chip reader. Other information needed for this screen, except for the OTP unique code, is available on the personal health card.

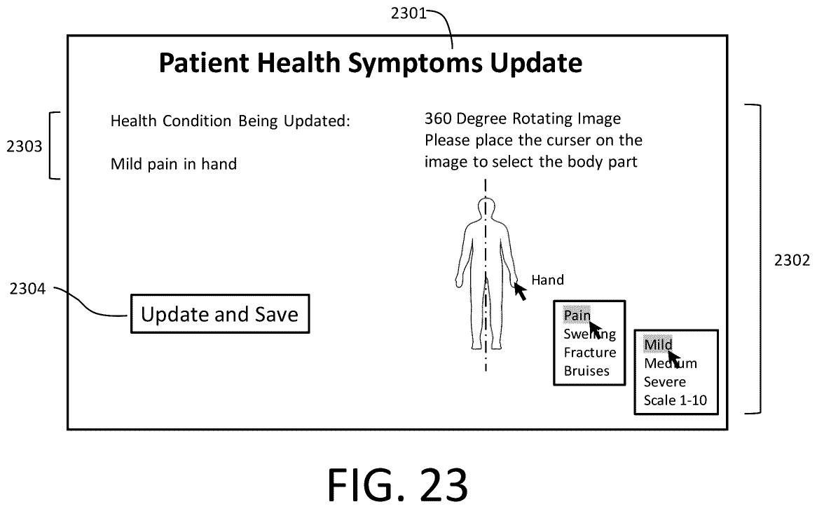

FIG. 23 is an example user health symptoms update page. A patient health symptoms update message 2301 is shown at the top of the web page to confirm that the correct page has been reached. This page includes a graphical feature 2302 for updating any health conditions the user is currently experiencing. A graphical representation of a body outline (male or female) provides for updating health conditions. A 360 degree rotating human image is provided which rotates by use of left/right arrow keys, or by clicking on the body outline and swiping left or right. When the cursor is hovered on the rotatable body outline, it displays that part of the body, which is click selectable by the user. Once the body part is selected a pop up menu displays a list of ailments associated with that body part. As shown in FIG. 7, pain, swelling, fracture, bruises are shown for a hand. The user then selects pain with the mouse. A second pop up menu appears, allowing the user to select the type of pain. The page then displays the health item selected in a text format 2303 for updating the online health record. The user then can update their record by clicking on an update and save button 2304.

A wide variety of other aliments can be logged this way. Various organs can be selected, and a zoom in of the body (not shown) can be added to better identify where a medical record should be recorded for an organ. For example, the liver, pancreas, stomach, small intestines, etc. are all in the abdomen and may be hard to select without the ability to zoom in.

If the user doesn't find a symptom/health conditions for a particular anatomical feature, the user could select an ‘other’ option (not shown) and manually enter it.

In general, a front and back outlined picture/animated human body is able to be displayed based on the gender of the registered user which they have selected while registering. It would also serve transgender registered users.

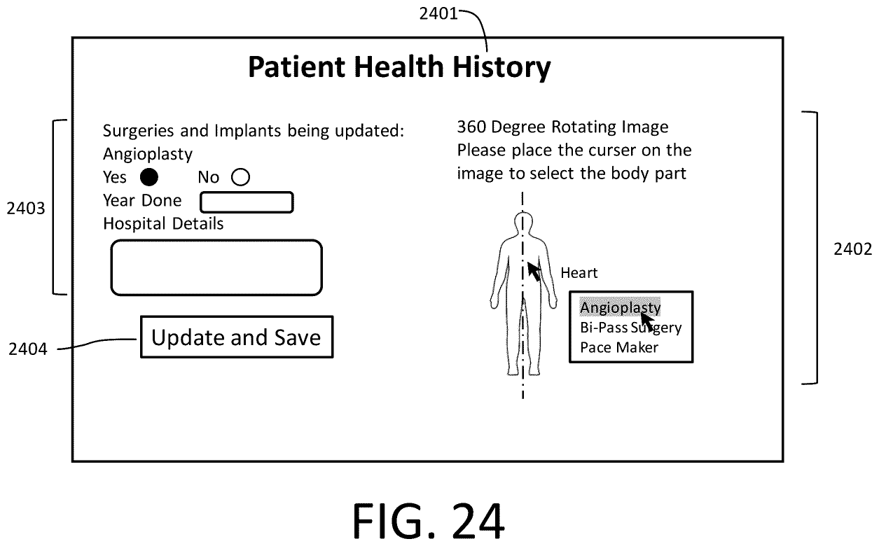

FIG. 24 is an example user past health history page. A patient health history message 2401 is shown at the top of the web page to confirm that the correct page has been reached.

Similarly to FIG. 23, a user friendly health history update page utilizes a graphical interface 2402. Again, a 360 degree rotating human image is provided. When the mouse pointer is hovered on a part of the rotating human body, it displays that part of the body. Once the body part is selected by clicking the mouse, a pop up menu displays a list of ailments previously recorded with that part. The user can select from a list in the pop up menu, that may in turn, become other pop-up menus, or alternately, an interactive text data base entry 2403 in the upper left of the screen.

The user then can update their record by clicking on an update and save button 2404. If the user doesn't find a medical procedure that they have gone through but not listed in the check list, then they could select an ‘other’ option (not shown) and manually enter and save it.

The user is able to delete any of the symptoms/health conditions that are incorrect. The user could make modifications easily and save it. Likewise, the registered user gives a background of their past health history in detail, like any surgery done in past, any implants that they have, any medicinal, latex, food or any other environmental allergies that they have, any over the counter or prescribed medication that they are taking on regular basis. The registered user can additionally give information on their health habits like smoking, alcohol, caffeine consumption, exercise etc.

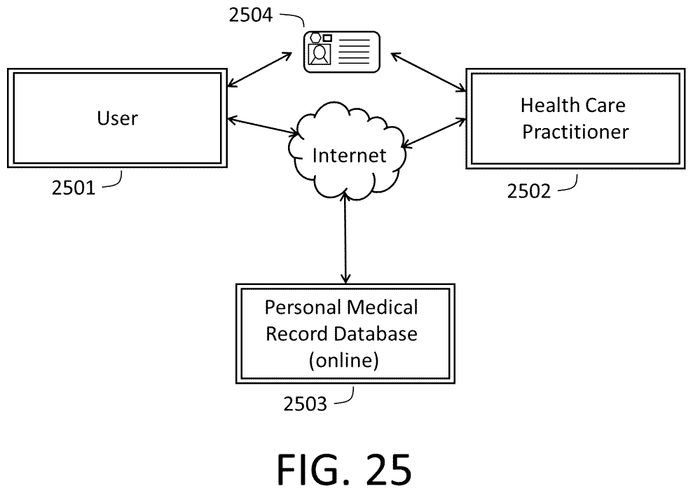

FIG. 25 is an abbreviated view of communication connections between the user, health care practitioner and the medical record database.

A user (i.e. patient) 2501 is connected to the internet by an internet connecting device, such as a computer, tablet, or phone. The user may use a number of methods and equipment for connecting to the internet including an internet service provider, a modem, and a wireless router; or by use of a cell phone connection to a cell phone tower. The user then, in turn, connects to an online personal medical record website 2503. The medical records website includes a medical record database, and associated web pages, to provide communication of the user's medical conditions, history, and symptoms.

The equipment by which a computer, tablet, or mobile phone connects to the internet, and access a remote web page, is well known technology and forms no part of the claimed invention.

Similarly, a health care provider 2502 connects to the internet, utilizing similar connecting equipment such as a computer, tablet, phone, mainframe computer. However, a specialized card scanning device is used to obtain a registration number from the personal health card 2504. The medical practitioner accesses the personal medical records database through the Internet, and utilizes the personal health card 2504 provided by the user 2501 to gain access.

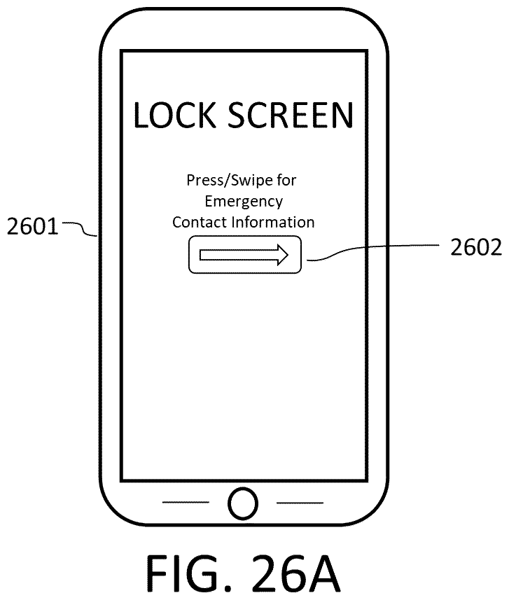

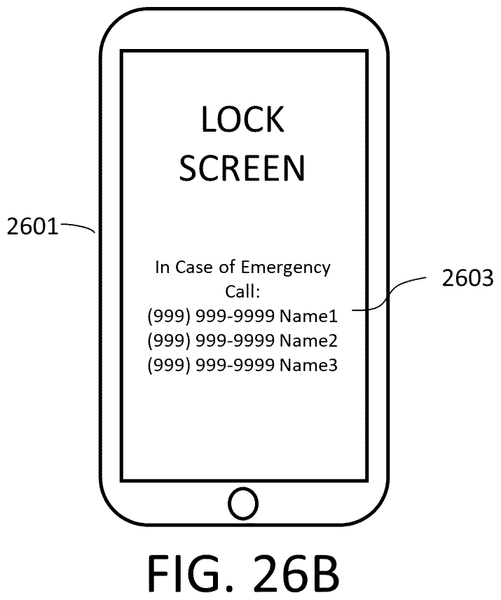

FIG. 26A-B is a view of a phone 2601 lock screen with an ‘in case of emergency’ (ICE) button 2602. Similarly, FIG. 26B also shows the phone 2601 after the button is activated where ICE contact name(s) and number(s) 2603 are listed. At least one contact name/number is displayed. Using a button 2602 (swipe or press) is important for privacy, so that the ICE information is not normally displayed on the lock screen, and the phone must be physically touched or held to obtain the ICE information. This provides important protection from anyone observing a name or phone number by merely looking at the lock screen. To add an ICE number, the phone owner adds an ICE reference in the contact information in their phone contact list.

For optional security, when the ICE button 2602 is activated, a name and contact phone number is first requested from the person accessing the ICE information, which is then logged in a phone access file. This provides the patient (or a family member) to verify that any ICE information access is legitimate by calling the access phone number in the access log file at a later time. If the name/number are not real, or a person answering the phone has no recollection of access, the patient will know that someone has been snooping and can take precautions.

ICE information is important for emergency responders which allows important medical communication about the person potentially being treated.

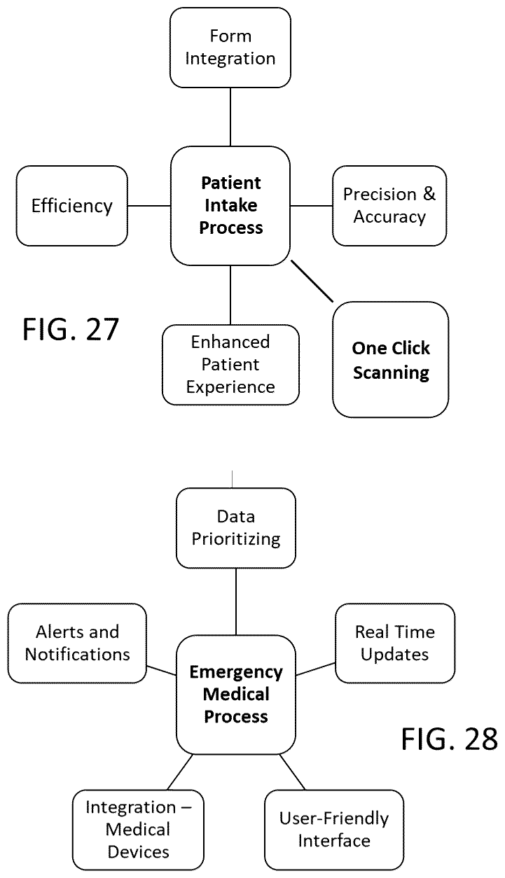

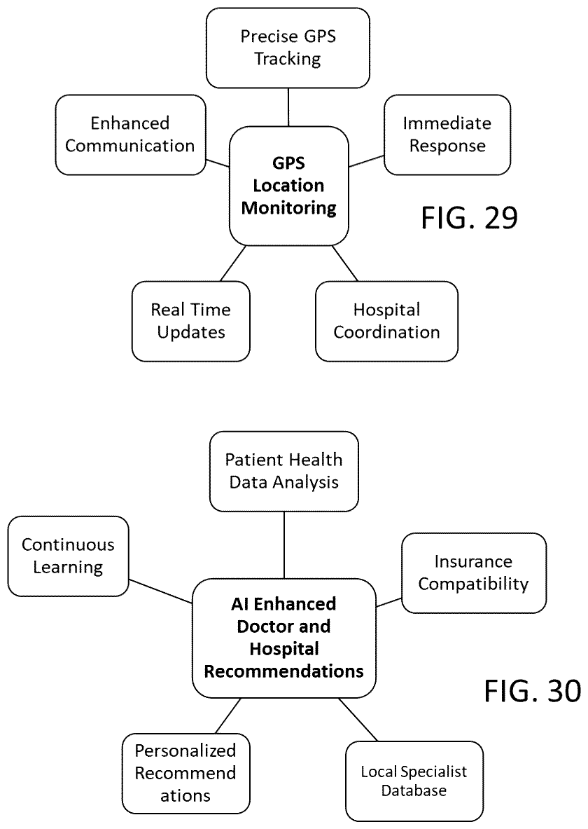

The AI Healthcare program (AI Healthcare) Streamlines the patient intake process for a new Healthcare Organization:
1. AI capable In-take Form (FIG. 27): The AI Healthcare streamlines the patient intake process by automatically populating patient intake forms with the necessary personal and health information. This will enhance operation efficiency, reduce manual errors, and provide better experience for both patients and healthcare providers. Key features are:
a. Form Integration: Regardless of the format or template used by different hospitals or clinics, the Healthcare AI can adapt to fill in the required fields accurately. This flexibility ensures that the Healthcare AI will work with any intake form, whether it is a digital form or a paper-based one that has been digitized.
b. Precision and Accuracy: By leveraging advanced algorithms and natural language processing (NLP), the AI ensures that all fields are filled out correctly, minimizing the risk of errors that can occur with manual data entry. This leads to more accurate patient records and better-informed healthcare providers.
c. Efficiency: Automating the intake process saves time for both patients and healthcare staff. Patients no longer need to repeatedly fill out forms, and staff can focus on more critical tasks rather than administrative work.
d. Enhanced Patient Experience: A smoother, faster intake process improves the overall patient experience, reducing wait times and ensuring that healthcare providers have all the necessary information upfront.
2. One-click Scanning: Healthcare AI uses one-click prescription scan to capture and store the medication information to the patient's profile. One-click prescription scan feature using AI-powered optical character recognition (OCR) automates the process of entering medication details accurately and reduces the risk of human error and saving time for both patients and healthcare providers. The system scans prescription labels, barcodes, and medication bottle details which has patient's name, age and drug dosage and securely stores to the patient's digital profile.
3. Prioritizing Patient Data: Healthcare AI is designed to enhance the efficiency and effectiveness of emergency medical care by prioritizing and sorting health symptoms and medication details based on the date. This ensures that paramedics and ER doctors have immediate access to the most recent and relevant information (FIG. 28).
a. Data prioritizing: The AI system automatically prioritizes health symptoms and medication details, placing the most recent entries at the top. This helps medical professionals quickly identify the latest health status and treatment history of the patient.
b. Real-Time Updates: As new health data is entered, the AI updates the records in real-time, ensuring that the information is always current. This is crucial in emergency situations where timely and accurate information can make a significant difference in patient outcomes.
c. User-Friendly Interface: The system is designed with an intuitive interface that allows paramedics and ER doctors to easily navigate through the patient's health records. This reduces the time spent on searching for information and allows more focus on patient care.
d. Integration with Medical Devices: Healthcare AI can integrate with various medical devices and electronic health records (EHR) systems, providing a comprehensive view of the patient's health status. This integration helps in making informed decisions quickly.
e. Alerts and Notifications: The AI can generate alerts and notifications for critical health changes or medication interactions, ensuring that medical professionals are immediately aware of any urgent issues.
4. GPS Location Patient Tracker (FIG. 29): Healthcare AI would precisely give the GPS location of the patient to the paramedics and the doctors. For example, how far is the patient from the hospital, within how many minutes will the patient be reaching the facility and be prepared to give the necessary medical treatment immediately. Healthcare Al's GPS functionality is designed to significantly enhance emergency response and hospital preparedness.
a. Precise GPS Tracking: The AI system continuously tracks the patient's location using GPS technology. This information is shared in real-time with paramedics and hospital staff.
b. Immediate Response: Paramedics receive the exact location of the patient, allowing them to reach the patient quickly and efficiently. This reduces response times and can be crucial in life-threatening situations.
c. Hospital Coordination: The system also provides the hospital with the patient's location, estimated time of arrival (ETA), and distance from the facility. This allows the hospital staff to prepare for the patient's arrival, ensuring that necessary medical treatments and resources are ready.
d. Real-Time Updates: As the patient is being transported, the AI continuously updates the ETA and location, providing both paramedics and hospital staff with the most current information. This helps in coordinating care and ensuring a smooth transition from the ambulance to the emergency room.
e. Enhanced Communication: The system can send alerts and notifications to the hospital's front office and medical team, keeping everyone informed about the patient's status and arrival time. This ensures that the medical team is prepared to act immediately upon the patient's arrival.
5. Alert Messaging System: Healthcare AI, is equipped with its sophisticated tool designed to assist healthcare professionals in various ways. One of its key features is its ability to alert doctors if they prescribe medications that a patient shouldn't take due to allergies or potential drug interactions. This is achieved through its advanced data processing capabilities, which analyze patient records and cross-references them with the prescribed medications to identity any potential risk. Additionally, our AI can help streamline administrative tasks, such as transcribing patient visits, generating notes, and filling out documents, which allows doctors to focus more on patient care. This integration of Al in healthcare not only enhances the efficiency of medical practices but also significantly improves patient safety by reducing the likelihood of medication errors
6. Enhanced hospital and doctor recommendation capabilities (FIG. 30): Healthcare AI is designed to enhance patient care by recommending local healthcare specialists tailored to a patient's specific health needs and medical insurance coverage.
a. Patient Health Data Analysis: The AI system analyzes the patient's health records, including medical history, current conditions, and any ongoing treatments. This helps in identifying the most suitable type of specialist required for the patient's condition.
b. Insurance Compatibility: The AI cross-references the patient's medical insurance details to ensure that the recommended specialists are within the insurance network. This helps in minimizing out-of-pocket expenses for the patient.
c. Local Specialist Database: Healthcare AI maintains an updated database of local healthcare specialists. This includes information on their areas of expertise, patient reviews, availability, and proximity to the patient's location.
d. Personalized Recommendations: Based on the analysis of health data and insurance compatibility, the AI provides a list of recommended specialists. These recommendations are personalized to ensure the best possible match for the patient's specific needs.
c. Continuous Learning: The AI system continuously learns from new data, patient feedback, and outcomes to improve its recommendations over time. This ensures that the system remains up to date with the latest medical practices and patient preferences.

By integrating these elements, Healthcare AI aims to streamline the process of finding the right healthcare specialist, making it easier and more efficient for patients to receive the care they need.

An AI Healthcare Algorithm is used for:
I. Significantly enhancing the process of capturing data from various commonly used file types, including images, text files, Word documents, and PDFs.
1. Image File processing

### Optical Character Recognition (OCR):

AI-powered OCR tools can recognize and extract text from images. This is useful for scanned documents, photos of text, and handwritten notes.
2. Text File processing

### Use Natural Language Processing (NLP):

Text Parsing: AI can parse and understand the structure of text files, extracting relevant information based on predefined rules or machine learning models.

Example: Leverage NLP libraries like spaCy or NLTK to extract specific data points from large text files, and integrate results in form fields.
3. MS Word File processing

Modeling: AI processes Word documents to extract structured data, such as tables, lists, and specific text patterns.
4. PDF File processing

Intelligent Data Capture: AI-Based Extraction: AI will extract text, tables, and images accurately from PDFs.
II. A high-level AI algorithm to capture data from a database and fill an intake form digitally involves several steps:
1. Data Extraction and confirmation: Query the Database: Use SQL or another query language to extract the necessary data from the database. Data Transformation: Convert the extracted data into a format suitable for the intake form. This involves cleaning, normalizing, and reformatting the data as needed.
2. Mapping Data to Form Fields:

Field Mapping: Create a map between the database fields and the intake form fields. This ensures that each piece of data is placed in the correct location on the form.

Validation Rules: Implement validation rules to ensure the data meets the required format and constraints for each form field.
3. Form Filling:

Automated Form Filling: Use a scripting language (e.g., Python, JavaScript) to automate the process of filling the form fields. Libraries like Selenium can simulate user input to fill web forms.

API Integration: If the intake form is part of a web application, AI uses APIs to directly place the data in the form fields.
4. Error Handling:

Error Detection: Implement mechanisms to detect and handle errors during data extraction, transformation, and form filling.

Logging and Alerts: Log errors will send alerts to notify users or administrators of any issues.
5. Algorithm Testing and Optimization:

Testing: Thoroughly test the algorithm with library data sets to ensure accuracy and reliability.

Optimization: Optimize the algorithm for performance and scalability, especially if dealing with large volumes of data.
III. Using AI to capture data from a mobile app and auto-populate an intake form to another system:
1. Optical Character Recognition (OCR): OCR technology can read and extract text from images or scanned documents. This is needed for capturing handwritten or printed information from forms, receipts, or other documents.
2. Natural Language Processing (NLP): NLP can interpret and process text data, making it possible to understand and extract relevant information from unstructured text inputs, such as notes or comments.
3. Machine Learning (ML): ML algorithms can be trained to recognize patterns and predict the correct fields for data entry. For example, an ML model can learn to identify and categorize different types of data (e.g., names, addresses, dates) and map them to the appropriate fields on an intake form.
4. Integration with APIs: AI systems can use APIs to communicate between the mobile app and the target system. This allows for seamless data transfer and real-time updates.
5. Intelligent Data Capture Systems: These systems use a combination of OCR, NLP, and ML to automatically identify and extract relevant data fields from various sources. They can validate the captured data to minimize errors and reduce manual intervention.
6. Open-Source Libraries: Tools like Tesseract OCR and OpenCV can be used to implement AI-driven data capture and form filling. These libraries provide pre-trained models and algorithms that can be customized for specific use cases.
IV. AI algorithm method to capture data from a database and fill in a form digitally:
1. Identify Data Requirements:

Determine the specific data fields required for the form. Map these fields to the corresponding form fields in personal database.
2. Data Extraction:

Use SQL queries to extract the necessary data from the database. Ensure data validation to maintain accuracy and consistency.
3. Data Transformation:

Format the extracted data to match the form's requirements. Handle any necessary data conversions (e.g., date formats, numerical precision).
4. Form Population:

Use a form-filling library or API to programmatically populate the form fields with the extracted data. Libraries like PDFBox for PDFs or HTML form libraries for web forms can be useful.
5. Error Handling:

Implement error-checking mechanisms to handle missing or incorrect data. Provide feedback or logging to track any issues during the process.
6. Submission:

Automate the form submission process if applicable. Ensure secure transmission of the form data, especially if it contains sensitive information.
7. Verification:

Verify that the form has been correctly filled and submitted. Implement a confirmation step to ensure data integrity.
V. AI algorithm steps for form filling from an uploaded image, by leveraging technologies like Optical Character Recognition (OCR) and machine learning:
1. Image Preprocessing:

Convert to Grayscale: Simplify the image by converting it to grayscale. Noise Reduction: Use filters to remove noise and improve text clarity: Binarization, by converting the image to black and white to enhance text recognition.
2. Optical Character Recognition (OCR):

Text Extraction: Use OCR tools like Tesseract to extract text from the image. This step converts the image text into machine-readable text. Text Segmentation: Break down the extracted text into individual characters, words, and lines.
3. Data Parsing and Structuring:

Field Identification: Use predefined templates or machine learning models to identify and classify different fields in the form (e.g., name, date, address). Data Formatting: Ensure the extracted data matches the required format for each field (e.g., date format, numerical values).
4. Data Validation and Correction:

Validation Rules: Apply rules to check the accuracy and completeness of the extracted data (e.g., valid date ranges, mandatory fields). Error Correction: use algorithms to correct common OCR errors (e.g., misrecognized characters).
5. Form Filling:

Field Mapping: map the extracted and validated data to the corresponding fields in the target form. Auto-Fill: programmatically fill the form fields with the extracted data.
6. Review and Confirmation:

Patient Review: have the patient or trained personnel review the filled form for accuracy. After acceptance submit the filled form to the desired healthcare organization.
VI. AI algorithm steps used for Data Validation
1. Type Checking: Ensure that the data is of the expected type (e.g., integers, strings, dates). Example: verify that a phone number field contains only numeric values.
2. Range Checking: Confirm that numerical values fall within a specified range. Example: ensure that an age field contains values between 0 and 120.
3. Format Checking: Validate that the data follows a specific format or pattern. Example: check that an email address contains an “@” symbol and a domain.
4. Consistency Checking: Ensure that related data fields are logically consistent. Example: verify that the start date is before the end date in a date range.
5. Uniqueness Checking: Ensure that certain fields contain unique values. Example: check that a user ID or email address is not already in use.
6. Presence Checking: Confirm that mandatory fields are not empty. Example: ensure that required fields like name and address are filled in.
7. Cross-Field Validation: Validate data by comparing multiple fields. Example: ensure that the total amount matches the sum of individual items.

Central computer system users, of varying kinds, such as patients, authorized users on behalf of patients, and health care providers all access the personal health website which resides on a database/central computer system at an internet accessible location. The central computer system users connect with the health care website through the internet utilizing their preferred hardware device as long as it has an internet browser incorporated in it. It does not matter what hardware is used and which path is taken through the Internet provided the user has a compatible web browser. The central computer system, where the personal health care website resides also interfaces with the Internet and manages the login and password information for the central computer system users. Browser software is used as the interface backbone for the users of the central computer system. A web browser (commonly referred to as a browser) is a software application for retrieving, presenting, and traversing information resources on the Internet, also called the World Wide Web. The central computer system uses specialized database software or highly customized software.

In an alternate embodiment, the central computer system resides on more than one computer at more than one geographic location. It is possible to have the user interface programming reside in one computer in one location, and the database reside in a second computer at a second location. The central computer system would generally include at least one CPU, transient and non-transient memory, and an operating system that would allow the CPU to be programmed to carry out a set of arithmetic or logical operations automatically. The parts of the computer do not have to be at one geographic location.

As used herein the terms central computer and computer system are intended to refer to a computer-related entity, comprising either hardware, a combination of hardware and software, software, or software in execution capable of performing the embodiments described. The disclosed embodiments which use the central computer refer to being interfaced to and controlled by a computer readable storage medium having stored thereon a computer program. The computer readable storage medium may include a plurality of components such as one or more of electronic components, hardware components, and/or computer software components. These components may include one or more computer readable storage media that generally store instructions such as software, firmware and/or assembly language for performing one or more portions of one or more implementations or embodiments of an algorithm as discussed herein. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or solid-state or electronic memory. Other forms of non-transitory and/or tangible computer readable storage media not list may be employed with the disclosed embodiments.

A number of such components can be combined or divided in an implementation of a computer system. Further, such components may include a set and/or series of computer instructions written in or implemented with any of a number of programming languages, as will be appreciated by those skilled in the art. Computer instructions are executed by at least one central processing unit. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

The embodied invention, as disclosed, makes use of data base driven by computer software to implement the steps necessary to implement a personal health website. Although users and humans' interface with the computer, the user interface along with steps take to register various entities, are the main features of the embodied invention.

Even though particular web pages are shown and described, some common web page features are not highlighted or mentioned. Common web browsing features such as a top menu system which allows for ready navigation of the website is not necessary to disclose. A button for returning to the home page is included on most web pages (even if not shown) as a disclosed embodiment, as well as back and next buttons as readily appreciated by those skilled in the art. Normally, the welcome/home web page is where the user begins his interface with the database, and often is the last page the user interfaces with.

While various embodiments of the present invention have been described, the invention may be modified and adapted to various operational methods to those skilled in the art. Therefore, this invention is not limited to the description and figure shown herein, and includes all such embodiments, changes, and modifications that are encompassed by the scope of the claims.

I claim:

1. A computer-implemented method for a personal healthcare record keeping system and a patient interface that provides updated and secured healthcare information to medical organizations, the method comprising:
A) providing a system comprising:
a) a storage server including a medical record database,
b) the medical record database having a plurality healthcare information stored therein,
c) a health card having either a readable magnetic strip or a readable microchip,
d) the health card configured to allow secure access to the healthcare information,
e) a patient interface to the medical record database,
f) the patient interface configured to have a display screen with a graphical user interface for editing and storing the healthcare information in the medical record database after access has been granted,
g) the graphical user interface includes a body image for medical data input,
h) an authorized user interface configured to allow access to a patient management interface, said patent management interference allows access to the healthcare information in the medical record database for creating and updating,
i) a date and time stamp log configured to record access to the authorized user interface,
h) a healthcare provider interface configured to allow secure access to the healthcare information,
i) an authorized user interface configured to allow secure access to the healthcare information,
B) utilizing the health card and the healthcare provider interface to obtain access to the healthcare information,
C) utilizing the health card and the patient interface to update the medical record database,
D) a healthcare program that incorporates sub programs comprising:
a) a user healthcare intake program,
b) a medical information scanning program for a camera image,
c) a prioritizing program which sorts medical history based on date,
d) a GPS program that facilitates communication about a user location,
e) a form assist program that prefills patient information input fields,
f) a data capture program obtaining medical information from:
documents made by word processing programs,
documents made by image processing programs, and
medical databases,
E) whereby the healthcare information accessed by the patient interface and is communicated to a healthcare organization.
